(19) United States
(12) Patent Application Publication
(10) Pub. No.: US 2015/0282990 A1
(43) Pub. Date: Oct. 8, 2015

(54) Inflatable Water Proof Ear Plug

(71) Applicant: Paul Krause, Scotts Valley, CA (US)

(72) Inventor: Paul Krause, Scotts Valley, CA (US)

(21) Appl. No.: 14/248,123

(22) Filed: Apr. 8, 2014

(51) Int. Cl. A61F 11/10

(52) U.S. Cl. CPC A61F 11/10

(57) ABSTRACT

This invention is an inflatable water proof ear plug which prevents moisture from entering the ear canal, reducing water intrusion and possible infection (“swimmer's ear”). A syringe or manual pump allows the user to inject air or fluid into the bladder inserted inside the outer ear canal, providing superior comfort and safety over the “one size fits all” ear plugs on the market. When the ear plug is positioned into the ear canal by inserting the syringe pump, it opens up a small check valve and a measured amount of air or liquid can be pumped into the expandable bladder. The air valve can be deflated for easy removal by pushing the small check valve tip. For diving extra measured amount of pressure can be injected to compensate for the increasing under water pressure.

### CROSS-REFERENCE TO RELATED APPLICATIONS

Application 20100071707 by inventor Wohl, Daniel whose External middle ear insufflation device partially looks similar to this invention, but its function is different. This invention tries to balance air pressure by pumping or sucking air in and out of the ear canal to balance the air pressure to the ear drum on air planes as it changes altitudes. U.S. Pat. No. 4,896,679 by St. Pierre shows and pneumatically expandable earplug without any functional pressure control device, making it functionally difficult to use to achieve proper fitting.

No Federal financial support was used for this patent application.

### BACKGROUND OF THE INVENTION

This invention is currently under US patent classification for Ear Plugs are U.S. Pat. Nos. CL381/329, 381/371 and 128/865, International CL. A61F 11/06 H04 R25/00. The specific existing problem is water intrusion into the ear canal during swimming with current ear plugs on the market.

This invention relates generally to the field of exterior ear protection from water intrusion to the ear drum. Water does not belong to the ear, during swimming, surfing, diving clean or polluted water may enter into the ear canal creating hearing problems. Water trapped in the ear canal not only causes discomfort it may create hearing loss. Trapped water provides the environment for fungal or bacterial growth that can lead to painful ear infections, or “swimmer's ear”, which usually requires medical treatment. People with ear drum perforations or holes in their eardrums hesitate to swim because water could enter through the eardrum into the middle ear, causing dizziness or a middle ear infection (“otitis media”). Children or adults who have had ear tubes surgically altered may have the same problems. Patients with congenitally small ear canals such as patients with Down's syndrome or acquired small ear canals (such as exostoses of the ear canal, or “surfer's ear”) are at increased risk of water trapping and ear infections.

Other patients who have had mastoidectomy surgery are told never to swim because their ears are impossible to dry completely afterwards. Children and people with either small or large ear canals struggle to find ear plugs that fit properly, do not fall out, or provide a true water proof seal. By inflating this invention the bladder does not create any air pressure differential by promoting air entry into the middle ear, no effect to the Eustachian Tube as the tympanic membrane remains stationary, there will be just some dead air behind the Inflatable Waterproof Ear Plug causing no pain nor any discomfort. This invention is a continuation of provisional Application No. 61/730,044 dated on Nov. 27, 2012.

### BRIEF SUMMARY OF THE INVENTION

The object of this invention is to create a flexible, proper fitting inflatable ear plug to prevent any water from flowing into the ear canal during swimming. The advantage of this invention is that it is adjustable due to the fact that it is inflatable, so it can provide a tight water proof seal. Other ear plugs currently on the market are squeezable foam what may even soak up the water, mainly usable for noise protection. Wax putty types may fall out, inconvenient to use, pieces may break off needing medical intervention to have it removed and it has a limited use. Reusable, flexible silicon ear plugs prevent water from flowing into an inner ear by using a flexible and thin peripheral flange does a better job keeping water out from the ear canal, but it is not adjustable, it requires special molding.

This Inflatable Water Proof Ear Plug should be able to solve this problem, being inflatable, it provides a snug comfortable water proof fit, it adjusts to any ear canal size, creates water tight protection and it could be used to block noise as well.

### BRIEF DESCRIPTION OF THE DRAWINGS

FIG. 1 on sheet 1 of 1 shows the complete ear plug section (1) of the invention what gets inserted into the outer ear canal deflated. It is made of flexible silicon or latex kind of material capable to be inflated and deflated through the valve assembly (2).

FIG. 2 on the same page shows the Inflatable Water Proof ear Plug adapter's (2) wax or silicon ring to provide additional support from wave actions as well as it provides more protection from splashing water. The proper operation/usage of this invention is to insert the inflating syringe (3.5) into the Inflatable Water Proof Ear Plug's adapter's intake section (14), slide it into the ear canal, inject the air or fluid for the proper pressure, than disengage the inflating syringe. Flexible wax, silicon or latex kind of ring around the check valve (2) helps stabilize the ear plug after insertion and provides additional seal from water splashing as well as noise protection. Cord (13) connects to the other ear plug wrapping around the neck to prevent loss.

### DETAILED DESCRIPTION OF THE INVENTION

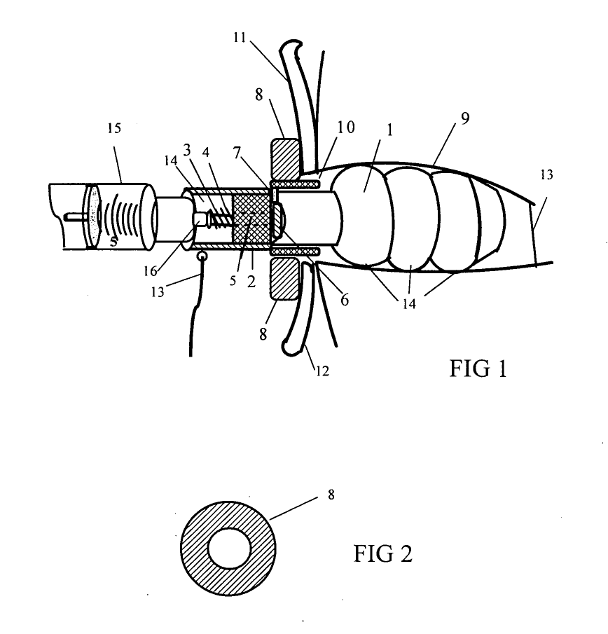

FIG. 1 on sheet 1 shows the complete ear plug assembly, the flexible and expandable insertion into the ear canal (9) leaving a secure air gap front of the ear Drum (15). The inflatable portion of the invention (1) what gets inserted into the outer ear canal when it is deflated.

It is made of flexible silicon or flexible rubber type material capable to be inflated and deflated through valve (4). The air intake into Ear Plug is tube (14), which is a slightly cone shaped inside or threaded for positive leak proof connection. The connecting air pump syringe (15) must be inserted, syringe tip (16) depresses spring loaded valve (3) for easier inflation process.

Adapter (2) trough insertion inlet (14) to enable the inflation and the deflation process trough air channel (5). At the time of the air-syringe insertion the center portion of the syringe pushes air valve (3) back by compressing the spring (4). Flexible rubber/silicon valve (6) normally is flat against the adapter's inner surface (7) holding it air tight with spring (4) to hold the inflatable section (1) sealed to keep the desired pressure locked in for the required duration. The inserted inflatable section of the flexible ear plug has at least three bulged out ridges (16) for better water blocking performance At time of deflation a gentle push at valve (3) deflates the inner balloon for easy removal. Cord (13) wraps around the neck to prevent loss.

FIG. 2 on the same page shows the Water Proof Inflatable Ear Plug adapter with a wax or soft silicon ring (8). This ring further holds the check valve assembly (2) in a stable position against wave actions as it is pressed against the outer ear surface (11) and gravity air pressure is holding it up. Left and right ear plugs may be connected through one flexible hose with an additional air intake and a flexible tube.

What is claimed is:

1. An ear canal insertable flexible Waterproof Inflatable Ear Plug comprising:
1. A dual valve for regulating the flow of air or fluid independently into the left and right ear canal, a housing which includes an interior adapter or ear plug chamber, inflow and outflow passages which fluidly communicate with the ear plug chamber and a valve seating surface disposed between the ear plug chamber, the outflow passage and the ear canal insertable inflatable section, syringe capable to insert air or fluid into the Ear Plug.
2. The inflatable section of this Waterproof Inflatable Ear Plug preferably has at least three expandable ridges for better sealing purposes providing multi stage water, blocking.
3. Above mentioned said device in claim one further has a circular wax, silicon, latex or similar flexible material wrapped around the ear plug chamber's outer side pressed against the outer ear to maximize water inflow prevention and to increase support to said device to prevent dislocation by waves.
4. By using an air/fluid syringe with measuring gage, consistent and different pressures could be achieved in left and right ears if it is needed for medical or diving reasons.
